(19) United States
(12) Patent Application Publication
(10) Pub. No.: US 2011/0028480 A1
(43) Pub. Date: Feb. 3, 2011

(54) ORODISPERSABLE FORMULATIONS OF PHOSPHODIESTERASE-5 (PDE-5) INHIBITORS

(75) Inventors: Adriana Rendon, Margate, FL (US); Esther Rendon, Margate, FL (US)

Correspondence Address: AKERMAN SENTERFITT, P.O. BOX 3188, WEST PALM BEACH, FL, 33402-3188, US

(73) Assignee: Ocean 1 806, LLC, Coral Gables, FL (US)

(21) Appl. No.: 12/935,796

(22) PCT Filed: Apr. 1, 2008
(86) PCT No.: PCT/US08/59057

§ 371 Date: Sep. 30, 2010

(51) Int. Cl. A61K 31/53; A61K 47/32; A61K 31/519; A61K 31/4985; A61P 15/00

(52) U.S. Cl. 514/243; 514/772.1; 514/252.16; 514/250

(57) ABSTRACT

Orodisperable formulations of phosphodiesterase-5 (PDE-5) inhibitors and methods of manufacturing such are provided. Further, an improved dosage form and method of treating sexual dysfunction are provided.

### BACKGROUND OF THE DISCLOSURE

(1) Field of the Disclosure

The disclosure generally relates to pharmaceutical formulations that comprise phosphodiesterase-5 (PDE-5) inhibitors.

(2) Description of Related Art

PDE-5 inhibitors including sildenafil, tadalafil, and vardenafil, are well known in the art. Certain formulations of PDE-5 inhibitors, such as the swallowable tablet dosage form of sildenafil citrate as used in Viagra® (Pfizer; New York, N.Y., USA), have been proven effective for erectile dysfunction. Vardenafil in Levitra® (Bayer; Leverkusen, Germany) and tadalafil in Cialis® (Eli Lilly; Indianapolis, Ind., USA), are also known to treat erectile dysfunction.

### SUMMARY OF THE DISCLOSED EMBODIMENTS

The instant application relates to a pharmaceutical formulation comprising a PDE-5 inhibitor and polacrilin potassium. One embodiment of the pharmaceutical formulation may be manufactured according to a sequential moist granulation procedure. A further embodiment of the invention may comprise a PDE-5 inhibitor, polacrilin potassium, and additional excipients and may be manufactured in accordance with a sequential moist granulation procedure. The formulation may be used to treat sexual dysfunction.

### BRIEF DESCRIPTION OF THE DRAWINGS

FIG. 1 reflects the percent dissolution over time of one embodiment of the disclosure as compared to percent dissolution over time of the 50 mg sildenafil citrate formulation of Viagra® (Pfizer; New York, N.Y., USA).

### DETAILED DESCRIPTION

The claimed embodiments relate to a novel pharmaceutical formulation that comprises a PDE-5 inhibitor as active agent and polacrilin potassium. One embodiment of the invention may additionally comprise additional excipients. A further embodiment may be manufactured in accordance with a sequential moist granulation process, in accordance with the method of manufacture presented herein. The disclosed formulations may be useful for treatment or prevention of medical disorders. Such disorders may include sexual dysfunction, such as erectile dysfunction or decreased blood flow to the pelvic area. Additionally, the disclosed formulation may be used to treat or prevent cardiovascular disorders. For instance, the disclosed formulation may be used to treat or prevent angina or hypertension, including pulmonary hypertension.

The formulation may be administered in a variety of dosage forms, such as a tablet, a tape as a thin strip that dissolves in a patient's mouth, a gum, or a chewable. A tablet may be intended for oral or vaginal administration. Tablets for oral administration comprising the formulation may be chewed, swallowed, or sucked upon so as to dissolve the tablet in a patient's mouth. In one embodiment, the tablet can dissolve without being chewed, and complete dissolution of the tablet in the mouth results in the fastest therapeutic effect. The PDE-5 active agent disperses as a result of exposure to saliva and enzymes in the mouth.

The PDE-5 inhibitor may be any PDE-5 inhibitor, such as sildenafil, vardenafil, tadalafil, or combinations thereof. The formulation may also include excipients in addition to polacrilin potassium. Polacrilin potassium in combination with the other disclosed ingredients masks a residual bitter taste of the active agent while concurrently increasing the dispersion, dissolution and therapeutic access or drug absorption of the formulation. Drug absorption refers to the process of drug movement from the site of administration into systemic circulation. Polacrilin potassium assists in the formulation-disintegrating process, due to the property of this component as a water-absorption agent.

The formulation has less micropore volume than other orodispersable formulations, increasing the formulation's hardness while decreasing the formulation's friability. The reduced micropore volume decreases the waste of raw materials in the manufacturing and packaging processes of the formulation. Conventional orodisperable tablets are low in hardness and highly porous to obtain dispersion times fast enough for an orodisperable formulation. The invention provides a new tablet characterized by compactness, resistance, smoothness, low micropore volume and high hardness that further exhibited high dissolution and dispersion capabilities.

Additional excipients may be included in the disclosed formulation. One embodiment of the formulation may additionally comprise a binding agent and a disintegrating agent. A binding agent is an ingredient that firmly ties together the various ingredients in the formulation. The binding agent will diminish the friability of the formulation and increment its hardness. A disintegrating agent is an ingredient that allows the formulation to disintegrate and release the active ingredient when the formulation is introduced into a dissolution medium. In such an embodiment comprising both a binding agent and a disintegrating agent, the binding agent may for example be mannitol, and the disintegrating agent may for example be croscarmellose sodium, crospovidone, sodium starch glycolate, or combinations thereof.

In one embodiment, the additional excipients further diminish the residual taste of the active agents. In a further embodiment, the additional excipients do not detract from the compressibility or texture of the formulations or the dissolution properties thereof.

In one embodiment, the formulation may include flavoring agents. Flavoring agents may be any such agents known in the art, such as conventional sweetening or masking agents, such as sugars, artificial sweeteners, non-sugar natural sweeteners, citric flavors, mints or menthols and specific bitter-taste masking agents, such as citric, malic or tartaric acids. Sugars may include, for example, sucrose, mannitol, sorbitol, and xylitol. Artificial sweeteners may include, for example, aspartame, and acesulfame. Non-sugar natural sweeteners may include, for example, estevia.

In one embodiment, the formulation may include a combination of mannitol, sucralose, powdered flavors and starch, such as a pregelatinized maize starch, e.g., 1500 Starch® (Colorcon; West Point, Pa., USA). The aforementioned combination of excipients further improves the compressibility of this embodiment of the formulation. Additionally, the aforementioned combination of excipients further increases dispersion speeds and enhances the taste-masking effect of polacrilin potassium. In a further embodiment, mannitol crystals may be included in the formulation, thereby achieving an additional improvement in the formulation's texture and compressibility.

Additionally, other excipients affecting texture, disintegration or dispersion of the formulation may be included. Such excipients include microcrystalline cellulose, croscarmellose sodium, crospovidone, silicon dioxide, sodium starch glycolate, and combinations thereof.

In another embodiment, the formulation may be in the dosage form of a vaginal tablet. Such an embodiment may include or omit colorants, sweeteners, and flavors. Such an embodiment may further include or omit menthol.

### EXAMPLES

Below are charts listing the ingredients of several formulations of the disclosed embodiments. Other formulations are possible. For instance, other binding agents, disintegrating agents, sweeteners, flavors and dyes may be used in place of those listed below. The microcrystalline cellulose used in the following formulations were in the form of particles having an average diameter of 190 μm as Avicel® PH-200 (FMC; Philadelphia, Pa., USA). Microcrystalline cellulose particles of other sizes may be used. The polacrilin potassium used in the below formulation was methacrylic acid polymer with divinylbenzene potassium salt, although other forms of polacrilin potassium may be used. Specifically, the polacrilin potassium used is the potassium salt of a unifunctional low-cross-linked carboxylic cation-exchange resin prepared from methacrylic acid and divinylbenzene. More specifically, the polacrilin potassium used was Tulsion® 339 (Thermax USA; Novi, Mich., USA) When previously dried at 105° C. for 6 hours, the polacrilin potassium used contains not less than 20.6% and not more than 25.1% of potassium. The polymer is linear, homogeneous, and comprises chains of equal lengths. The polymer comprises combinations of methacrylic acid and divinylbenzene. A monograph of the polacrilin potassium polymer used is presented below:

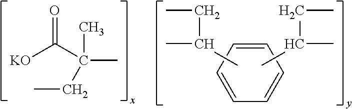

TABLE 1
Sildenafil Formulations

| | | Formula No. 1; | | |
|---|---|---|---|---|
| | | Preferred | | |
| | | Formula: | Formula No. 2: | Formula No. 3: |
| | Weight Range | Weight as (%) | Weight as (%) | Weight as (%) |
| | as (%) of Total | of Total | of Total | of Total |
| Ingredient | Weight | Weight | Weight | Weight |
| Sildenafil Citrate | 1 to 50 | 19.89 | 19.93 | 22.62 |
| Polacrilin potasium | 10 to 50 | 24.07 | 15.95 | 9.20 |
| Color blue FD&C No. 2 | 0.1 to 0.5 | 0.10 | 0.20 | 0.20 |
| lake | | | | |
| Manitol | 10 to 40 | 28.72 | 35.08 | 35.55 |
| Microcrystalline cellulose | 10 to 30 | 9.62 | 11.35 | 12.10 |
| Sucralose | 0.1 to 3 | 0.96 | 0.96 | 0.75 |
| Croscarmellose sodium | 0.5 to 4 | 2.83 | 2.89 | 3.10 |
| Cream flavor | 0.1 to 2 | 0.34 | 0.34 | 0.40 |
| Mint flavor | 0.5 to 4 | 2.26 | 2.01 | 3.50 |
| Crospovidone | 0.5 to 2 | 1.93 | 1.93 | 2.10 |
| Sodium Starch Glycolate | 2 to 10 | 2.83 | 2.89 | 4.00 |
| Magnesium stearate | 0.5 to 5 | 2.93 | 2.93 | 3.10 |
| Colloidal silicon dioxide | 0.1 to 4 | 0.48 | 0.48 | 0.32 |
| Menthol | 0.1 to 5.0 | 1.50 | 1.50 | 1.50 |
| Talc | 0.1 to 3.0 | 1.56 | 1.56 | 1.56 |
| TOTAL | | 100.00 | 100.00 | 100.00 |

TABLE 2
Vardenafil Formulations

| | | Formula No. 1; | | |
|---|---|---|---|---|
| | | Preferred | | |
| | | Formula: | FORMULA No. 2: | FORMULA No. 3: |
| | Weight Range | Weight as (%) | Weight as (%) | Weight as (%) |
| | as (%) of Total | of Total | of Total | of Total |
| Ingredient | Weight | Weight | Weight | Weight |
| Vardenafil | 1 to 50 | 5.20 | 4.95 | 4.66 |
| Polacrilin potasium | 10 to 50 | 23.00 | 21.00 | 19.10 |
| Yellow Iron Oxide | 0.1 to 0.5 | 0.20 | 0.22 | 0.30 |
| Manitol | 10 to 40 | 36.10 | 37.68 | 39.00 |
| Microcrystalline cellulose | 10 to 30 | 22.12 | 23.00 | 22.64 |
| Sucralose | 0.1 to 3 | 0.10 | 0.15 | 0.20 |
| Croscarmellose sodium | 0.5 to 4 | 3.00 | 2.50 | 3.50 |
| Cream flavor | 0.1 to 2 | 0.20 | 0.25 | 0.30 |
| Mint flavor | 0.5 to 4 | 0.30 | 0.35 | 0.40 |
| Crospovidone | 0.5 to 2 | 1.80 | 2.00 | 1.90 |
| Sodium starch glicolate | 2 to 10 | 4.50 | 4.60 | 4.70 |
| Magnesium stearate | 0.5 to 5 | 0.98 | 1.00 | 1.00 |
| Colloidal silicon dioxide | 0.1 to 4 | 0.40 | 0.30 | 0.30 |
| Menthol | 0.1 to 5.0 | 1.50 | 1.50 | 1.50 |
| Talc | 0.1 to 3.0 | 0.60 | 0.50 | 0.50 |
| TOTAL | | 100.00 | 100.00 | 100.00 |

TABLE 3
Tadalafil Formulations

| | | FORMULA No. 1: | FORMULA No. 2: | FORMULA No. 3: |
|---|---|---|---|---|
| | Weight Range | Weight as (%) | Weight as (%) | Weight as (%) |
| | as (%) of Total | of Total | of Total | of Total |
| Ingredient | Weight | Weight | Weight | Weight |
| Tadalafil | 1 to 50 | 7.50 | 7.30 | 7.10 |
| Polacrilin potasium | 10 to 50 | 21.00 | 25.00 | 20.00 |
| Yellow Iron Oxide | 0.1 to 0.5 | 0.30 | 0.40 | 0.50 |
| Manitol | 10 to 40 | 39.00 | 38.00 | 39.00 |
| Microcrystalline cellulose | 10 to 30 | 19.32 | 16.53 | 20.87 |
| Sucralose | 0.1 to 3 | 0.30 | 0.33 | 0.25 |
| Croscarmellose sodium | 0.5 to 4 | 2.00 | 3.00 | 2.50 |
| Cream flavor | 0.1 to 2 | 0.30 | 0.31 | 0.33 |
| Mint flavor | 0.5 to 4 | 0.30 | 0.33 | 0.40 |
| Crospovidone | 0.5 to 2 | 1.50 | 1.50 | 1.40 |
| Sodium starch glicolate | 2 to 10 | 4.50 | 4.00 | 4.10 |
| Magnesium stearate | 0.5 to 5 | 1.20 | 1.00 | 1.10 |
| Colloidal silicon dioxide | 0.1 to 4 | 0.30 | 0.30 | 0.35 |
| Menthol | 0.1 to 5.0 | 1.50 | 1.50 | 1.50 |
| Talc | 0.1 to 3.0 | 0.98 | 0.50 | 0.60 |
| TOTAL | | 100.00 | 100.00 | 100.00 |

An example of quantities of ingredients per tablet according to Formula 1 of the Sildenafil Citrate formulation presented in Table 1 is presented below. Mannitol particles having an average particle diameter of 300 μm as Mannitol 300 (Merck Colombia S.A; Bogota, Colombia) were used in the below formulation. The Irish cream flavor utilized in the below formulation was Irish Cream Durarome® (Firmenich; Geneva, Switzerland).

TABLE 4
Sildenafil Citrate Formula 1 Ingredients Per Tablet

| | Weight as (%) of | |
|---|---|---|
| Ingredient | Total Weight | mg/Tablet |
| Sildenafil Citrate | 19.89% | 70.23 |
| Polacrilin in potasium | 24.07% | 84.97 |
| Color blue FD&C No. 2 lake | 0.10% | 0.35 |
| Manitol | 28.72% | 101.40 |
| Microcrystalline cellulose | 9.62% | 33.96 |
| Sucralose | 0.96% | 3.38 |
| Croscarmellose sodium | 2.83% | 9.99 |
| Cream flavor | 0.34% | 1.19 |
| Mint flavor | 2.26% | 7.98 |
| Crospovidone | 1.93% | 6.80 |
| Sodium Starch Glycolate | 2.83% | 9.99 |
| Magnesium stearate | 2.93% | 10.34 |
| Colloidal silicon dioxide | 0.48% | 1.70 |
| Menthol | 1.50% | 5.30 |
| Talc | 1.56% | 5.50 |
| TOTAL | 100.00% | 353.08 |

Comparative dissolution tests of the aforementioned Formula 1 of the sildenafil citrate formulation, and of Viagra® (Pfizer; New York, N.Y., USA) having an equivalent amount of sildenafil citrate were performed. The comparative dissolution tests were executed under standard conditions, wherein standard conditions are defined as conditions in an approximately 900 mL solution comprising HCl 0.01 N, at a temperature of approximately 37° C. while rotated at a speed of approximately 50 RPM. The comparative dissolution tests were executed in a palette SR8 dissoluter apparatus (Hanson Research; Chatsworth, Calif., USA). The following percentages and dissolution timing were found:

TABLE 5
Dissolution Percentages of Sildenafil Citrate Formula 1

| Sildenafil | TIME (MINUTES) | | | | | | |
|---|---|---|---|---|---|---|---|
| Citrate | Dissolved Percentage of Sildenafil | | | | | | |
| Formula 1 | 0.0 | 0.5 | 1.0 | 1.5 | 2.0 | 5.0 | 8.0 |
| Sample 1 | 0 | 62 | 85 | 89 | 100 | 99 | 99 |
| Sample 2 | 0 | 62 | 96 | 98 | 99 | 99 | 99 |
| Sample 3 | 0 | 81 | 95 | 96 | 100 | 99 | 100 |
| Sample 4 | 0 | 75 | 91 | 92 | 98 | 98 | 101 |
| Sample 5 | 0 | 70 | 82 | 98 | 99 | 99 | 99 |
| Sample 6 | 0 | 75 | 85 | 85 | 97 | 99 | 98 |
| AVERAGE | 0 | 71 | 89 | 93 | 99 | 99 | 99 |
| Standard | 0.00 | 7.68 | 5.83 | 5.29 | 1.17 | 0.41 | 1.03 |
| Deviation | | | | | | | |
| Relative | | 10.8 | 6.6 | 5.7 | 1.2 | 0.4 | 1.0 |
| Standard | | | | | | | |
| Deviation | | | | | | | |
| (%) | | | | | | | |

TABLE 6
Dissolution Percentages of Viagra ® (Pfizer; New York, NY, USA)
50 mg Sildenafil ®

| | TIME (MINUTES) | | | | | | |
|---|---|---|---|---|---|---|---|
| Viagra ® | Dissolved Percentage of Sildenafil | | | | | | |
| 50 mg | 0.0 | 0.5 | 1.0 | 1.5 | 2.0 | 5.0 | 8.0 |
| Sample 1 | 0.00 | 0.00 | 0.00 | 21.00 | 38.00 | 62.00 | 77.00 |
| Sample 2 | 0.00 | 0.00 | 0.00 | 21.00 | 47.00 | 64.00 | 86.00 |
| Sample 3 | 0.00 | 0.00 | 0.00 | 26.00 | 48.00 | 55.00 | 62.00 |
| Sample 4 | 0.00 | 0.00 | 0.00 | 25.00 | 48.00 | 65.00 | 79.00 |
| Sample 5 | 0.00 | 0.00 | 0.00 | 10.00 | 31.00 | 49.00 | 61.00 |
| Sample 6 | 0.00 | 0.00 | 0.00 | 17.00 | 47.00 | 70.00 | 79.00 |
| AVERAGE | 0.00 | 0.00 | 0.00 | 20.00 | 43.17 | 60.83 | 74.00 |
| Standard | 0.00 | 0.00 | 0.00 | 5.87 | 7.08 | 7.57 | 10.16 |
| Deviation | | | | | | | |
| Relative | 0.00 | 0.00 | 0.00 | 29.33 | 16.41 | 12.45 | 13.73 |
| Standard | | | | | | | |
| Deviation | | | | | | | |
| (%) | | | | | | | |

TABLE 7
Dissolution Comparative Results of Sildenafil Citrate Formula 1,
with Viagra ® 50 mg (Pfizer; New York, NY, USA)

| | TIME (MINUTES) | | | | | | |
|---|---|---|---|---|---|---|---|
| | Average Dissolved Percentage of Sildenafil | | | | | | |
| SAMPLE | 0.0 | 0.5 | 1.0 | 1.5 | 2.0 | 5.0 | 8.0 |
| Sildenafil | 0.0 | 70.8 | 89.0 | 93.0 | 98.8 | 98.8 | 99.3 |
| Citrate | | | | | | | |
| Formula 1 | | | | | | | |
| VIAGRA | 0.0 | 0.0 | 0.0 | 20.0 | 43.2 | 60.8 | 74.0 |
| 50 mg | | | | | | | |

Table 7 illustrates that the preferred formulation of sildenafil citrate, as provided in Formula 1, exhibits dissolution of the active agent of at least 90% within 90 seconds, and at least 95% within 120 seconds.

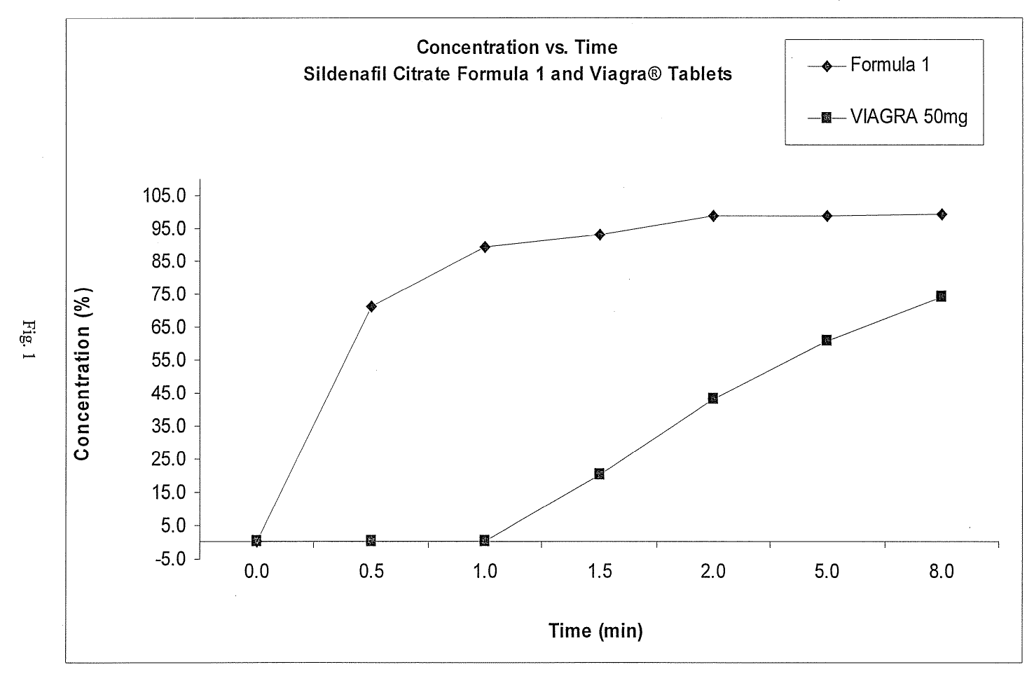

FIG. 1 is a graph reflecting the results provided in Table 7. The tests performed and the corresponding comparative curves show that the preferred embodiment of the sildenafil citrate version of the formulation, as described as the sildenafil citrate Formula 1, presents faster dissolution values than those presented by conventional solid formulations of sildenafil citrate, such as Viagra® (Pfizer; New York, N.Y., USA). Like to results shown in FIG. 1, the results of such further analyses evidence a notable improvement in dissolution speed as compared to conventional PDE-5 inhibitor tablets.

### Method of Manufacture

Although other protocols may be utilized, the following method was used to prepare the disclosed formulations.

Granulation and drying is the first step in the manufacturing process. During this step, polacrilin potassium acts as an ion exchange resin with the PDE-5 inhibitor, resulting in a masking of the bitter taste of the PDE-5 inhibitor.

In practice, a suspension was prepared by mixing ethyl alcohol, water and polacrilin potassium. As an example, polacrilin potassium as methacrylic acid polymer with divinylbenzene potassium salt was used. Specifically, an amount of ethyl alcohol and water were combined such that the ratio when combined with the PDE-5 inhibitor would be 1:1:1.25 parts by weight water: ethyl alcohol: PDE-5 inhibitor. The alcohol, water, and polacrilin potassium in an amount as called for by the desired formulation table were mixed while being stirred constantly at about 15 RPM for 15 minutes, although other percentages, speeds and times may be used. Then, approximately half of the quantity called for by the formulation of the pharmaceutically acceptable colorants was added to the mixture. For example, the blue dye FD&C No. 2 lake was used. Some other fraction of the colorants may be added. Additional colorants, replacement colorants, or no colorants may also be incorporated into the mixture.

Next, the PDE-5 inhibitor was added to the polacrilin potassium mixture, which was then stirred, dried and sieved. Stirring took place at approximately 15 RPM and was maintained for about 20 minutes, although other durations of mixing and speeds of mixing may be used. The resulting granular mass was dried in a tray drying cabinet at approximately 55° C. until the humidity within the cabinet registered at between approximately 2% and approximately 3%. The active ingredient-comprising granular mass was then passed through a sieve with mesh #20, although other sizes of mesh may be used.

Next, the remaining half of the blue colorant or dye in the sildenafil citrate formula I was dissolved with menthol in the remaining amount of ethyl alcohol.

Subsequently, a mixture of the excipients was prepared. The various excipients were sieved and mixed, and the resulting mixture was then dried and sieved. The following ingredients in amounts according to the desired formulation table were passed through a sieve with mesh #20, although other sizes of mesh may be used, and placed on a planetary mixer: mannitol crystals, croscarmellose sodium, microcrystalline cellulose, sucralose, mint flavor, and the Irish cream flavor. The flavoring elements may be omitted, particularly as dependent on the desired method of administration. The mixture on the planetary mixer was then granulated with the menthol, blue colorant and ethyl alcohol solution. The resulting granular mass was dried in a tray drying cabinet at approximately 55° C. until the humidity of the cabinet reached between about 2% and about 3%. The excipient granular mass was then passed through a sieve with mesh #20, although other mesh sizes may be utilized.

Following granulation and drying, the PDE-5-comprising granular mass and the excipient granular mass were then combined. The PDE-5 comprising granular mass and excipient granular mass were mixed in a planetary mixer for approximately 15 minutes at about 20 RPM to achieve a powder mass.

Then, additional excipients were sieved and mixed with the powder mass. The following ingredients listed in the desired formulation table were passed through a sieve with mesh #12, although other sizes of mesh may be used: sodium starch glycolate, crospovidone and colloidal silicon dioxide. After being passed through the sieve, the aforementioned ingredients were then added to the powder mass in the planetary mixer. The mixture was continued to be mixed at 20 RPM for approximately 5 minutes.

Following mixing, steps of lubrication and compression were undertaken. The magnesium stearate and the talc from the desired formulation ingredient list were then passed through a sieve with mesh #12, although other sizes of mesh may be used. The talc and magnesium stearate was then added to the powder mass in the planetary mixer. The ingredients in the planetary mixer were then continued to be mixed at 20 RPM for approximately 5 minutes. The resulting granular mass was then weighed for efficiency purposes and the proceeded to a compression process. The formulation may be prepared in a variety of dosage forms, such as a tablet, a tape, a gum, or a chewable. If prepared as a tablet, the final tablet hardness and weight ranges should be: 2.0 to 6.0 Kp. and 353 to 367 mg.

1. A pharmaceutical formulation comprising a PDE-5 inhibitor in an amount from 1% to 50% of the total weight of the formulation and polacrilin potassium in an amount from 5% to 50% of the total weight of the formulation,
wherein the formulation is provided in an orodispersable form and the polacrilin potassium acts at least in part to mask a residual bitter taste of the PDE-5 inhibitor.

2. (canceled)

3. The formulation as in claim 2, wherein the PDE-5 inhibitor comprises from 4.66% to 22.62% of the total weight of the formulation and wherein polacrilin potassium comprises 9.2% to 25% of the total weight of the formulation.

4. The formulation as in claim 1, wherein said formulation under standard conditions exhibits dissolution of the active agent of at least 90% within 90 seconds, and at least 95% within 120 seconds.

5. The formulation as in claim 1, further comprising at least one binder and at least one disintegrating agent, wherein said at least one binder comprises from 10% to 40% total weight of said formulation, and wherein said at least one disintegrating agent comprises from 3% to 16% the total weight of the formulation.

6. The formulation as in claim 5, wherein the at least one binder is mannitol and wherein the at least one disintegrating agent is croscarmelose sodium, crospovidone, sodium starch glycolate, or combinations thereof.

7. The formulation as in claim 1, wherein the PDE-5 inhibitor is sildenafil citrate.

8. The formulation as in claim 7, comprising a component selected from the group consisting of:
a) Mannitol;
b) Pharmaceutically acceptable colorants;
c) Microcrystalline cellulose;
d) Sucralose;
e) Croscarmellose sodium;
f) Pharmaceutically acceptable flavor;
g) Crospovidone;
h) Sodium starch glycolate;
i) Magnesium stearate;
j) Coloidal Silicon dioxide;
k) Menthol;
l) Talc; and
m) Combinations thereof.

9. The formulation as in claim 8, comprising as a percentage of the total weight of the formulation:
a) Pharmaceutically acceptable colorants from 0.1% to 0.5%;
b) Mannitol from 10% to 40%;
c) Microcrystalline cellulose from 10% to 30%;
d) Sucralose from 0.1% to 3%;
e) Croscarmellose sodium from 0.5% to 4%;
f) Pharmaceutically acceptable flavor from 0.6% to 6%;
g) Crospovidone from 0.5% to 2%;
h) Sodium starch glycolate from 2% to 10%;
i) Magnesium stearate from 0.5% to 5%;
j) Coloidal Silicon dioxide from 0.1% to 4%;
k) Menthol from 0.1 to 5.0%; and
l) Talc from 0.1% to 3.0%.

10. The formulation as in claim 9, wherein sildenafil citrate comprises 20% of the formulation and polacrilin potassium comprises 24% of the formulation.

11. The formulation as in claim 1, wherein the PDE-5 inhibitor is vardenafil.

12. The formulation as in claim 11, comprising a component selected from the group consisting of:
a) Mannitol;
b) Pharmaceutically acceptable colorant;
c) Microcrystalline cellulose;
d) Sucralose;
e) Croscarmellose sodium;
f) Pharmaceutically acceptable flavor;
g) Crospovidone;
h) Lactose monohydrate;
i) Pregelatinized starch;
j) Sodium starch glycolate;
k) Magnesium stearate;
l) Coloidal silicon dioxide;
m) Menthol
n) Talc; and
o) Combinations thereof.

13. The formulation as in claim 12, comprising as a percentage of the total weight of the formulation:
a) Mannitol from 10% and 40%;
b) Pharmaceutically acceptable colorants from 0.1% to 0.5%;
c) Microcrystalline cellulose from 10% to 30%;
d) Sucralose from 0.1% to 3%;
e) Croscarmellose sodium from 0.5% to 4%;
f) Pharmaceutically acceptable flavor from 0.6% to 6%;
g) Crospovidone from 0.5% to 2%;
h) Sodium starch glycolate from 2% to 10%;
i) Magnesium stearate from 0.5% to 5%;
j) Coloidal silicon dioxide from 0.1% to 4%;
k) Menthol from 0.1% to 5.0%; and
l) Talc from 0.1% to 3%.

14. The formulation as in claim 13, wherein vardenafil comprises 5.2% of the total weight, and polacrilin potassium comprises 23% of the total weight.

15. The formulation as in claim 1, wherein the PDE-5 inhibitor is tadalafil.

16. The formulation as in claim 15, comprising a component selected from the group consisting of:
a) Mannitol;
b) Pharmaceutically acceptable colorants;
c) Microcrystalline cellulose;
d) Sucralose;
e) Croscarmellose sodium;
f) Pharmaceutically acceptable flavor;
g) Crospovidone;
h) Lactose monohydrate;
i) Pregelatinized starch;
j) Sodium starch glycolate;
k) Magnesium stearate;
l) Coloidal silicon dioxide;
m) Talc;
n) Menthol; and
o) Combinations thereof.

17. The formulation as in claim 16, comprising as a percentage of the total weight of the formulation:
a) Mannitol from 10% to 40%;
b) Pharmaceutically acceptable colorants from 0.1% to 0.5%;
c) Microcrystalline cellulose from 10% to 30%;
d) Sucralose from 0.1% to 3%;
e) Croscarmellose sodium from 0.5% to 4%;
f) Pharmaceutically acceptable flavor from 0.1% to 6%;
g) Crospovidone from 0.5% to 2%;
h) Sodium starch glycolate from 2% to 10%;
i) Magnesium stearate from 0.5% to 5%;
j) Coloidal Silicon dioxide from 0.1% to 4%;
k) Menthol 0.1% to 5.0%; and
l) Talc from 0.1% to 3%.

18. The formulation of claim 17, wherein tadalafil comprises 7.5% the total weight of the formulation, and polacrilin potassium comprises 21% the total weight of the formulation.

19. A method of manufacturing a formulation comprising a pharmaceutically effective amount of a PDE-5 inhibitor and polacrilin potassium, comprising mixing the polacrilin potassium with PDE-5 inhibitor for at least ten minutes.

20. The method of claim 19 wherein the polacrilin potassium is first mixed with water and alcohol prior to being mixed with the PDE-5 inhibitor.

21. The method of claim 19, wherein the polacrilin potassium is in the form of methacrylic acid polymer with divinylbenzene potassium salt.

22. A method of treating sexual dysfunction comprising the steps of:
providing a formulation comprising a pharmaceutically effective amount of from 1% to 50% of the total weight of the formulation of a PDE-5 inhibitor and polacrilin potassium in an amount from 5% to 50% of the total weight of the formulation, wherein the polacrilin potassium acts at least in part to mask a residual bitter taste of the PDE-5 inhibitor; and
administering the formulation in an orodispersable form.

23. The method of claim 22, wherein the PDE-5 inhibitor comprises from 4.66% to 22.62% of the total weight of the formulation and wherein polacrilin potassium comprises 9.2% to 25% of the total weight of the formulation.

24. The method of claim 23, wherein the total weight of the formulation is from 353 mg and 367 mg.
